(19) United States
(12) Patent Application Publication
(10) Pub. No.: US 2016/0066997 A1
(43) Pub. Date: Mar. 10, 2016

(54) Surgical Instrument Organizer and Methods Therefor

(71) Applicant: Medline Industries, Inc., Mundelein, IL (US)

(72) Inventors: Xingquan Ren, Skokie, IL (US); Wesley Gray Hardin, Huntsville, AL (US); Magda Zavala, Gurnee, IL (US)

(21) Appl. No.: 14/481,206

(22) Filed: Sep. 9, 2014

(51) Int. Cl. A61B 19/02; F16M 13/00

(52) U.S. Cl. CPC A61B 19/0256; F16M 13/00; A61B 2019/0201

(57) ABSTRACT

A surgical instrument organizer (400) includes an elongated body (401) and a flexible strip (402) coupled to the elongated body. The flexible strip is bendable to retain the elongated body in a contoured configuration defined by one or more bends of the flexible strip. Surgical instruments (301) can be placed within the folds. The surgical instrument organizer can be formed to accommodate a specific number of instruments corresponding to a specific surgical procedure.

### BACKGROUND

1. Technical Field

This disclosure relates generally to organizers, and more particularly to surgical instrument organizers.

2. Background Art

Surgical instruments are used in medical procedures. Such instruments can include scissors, syringes, scalpels, blades, shavers, catheter needles, trocars, and other items. When surgery begins, a health care services provider sometimes arranges the various instruments that will be needed in surgery on a table or “Mayo” stand. Conventionally, a health care services provider places a towel on the stand, and then rolls another towel into a long “jellyroll” shape. Surgical instruments can then be arranged along the jellyroll. This placement attempts to elevate graspable portions of each surgical instrument. For example, the health care services provider may put scissors on the jellyroll with the point on the stand and the handle loops propped in the air by the jellyroll.

This ad hoc organizing technique suffers from problems. First, most surgical instruments flop over on their sides along the jellyroll, which makes grasping the graspable portions difficult. This problem is exacerbated when, for example, a pair of scissors is toppled onto a scalpel, as a health care services provider reaching for the scissors runs the risk of being cut by the scalpel. This “bump” problem can also result in difficulty in identifying which instrument is atop the other, which delays the procedure while the healthcare services provider sorts out which instrument is which. Second, surgical instruments disposed along the jellyroll can be bumped, jostled, or even knocked off the stand. When this occurs, the preparation procedure may need to start anew due to the sterile field being compromised. Either problem creates delay and causes confusion during the surgical procedure.

It would be advantageous to have an improved surgical instrument organizer. Even more advantageous would be an inexpensive, improved surgical instrument organizer that reliably held surgical instruments for ease of identification and grasping.

### BRIEF DESCRIPTION OF THE DRAWINGS

FIG. 1 illustrates a prior art towel being rolled into a jellyroll.

FIG. 2 illustrates a health care services provider unsuccessfully attempting to shape a prior art towel jellyroll.

FIG. 3 illustrates a prior art towel jellyroll used as a surgical instrument organizer.

FIG. 4 illustrates an explanatory surgical instrument organizer configured in accordance with one or more embodiments of the disclosure.

FIG. 5 illustrates one explanatory flexible strip in accordance with one or more embodiments of the disclosure.

FIG. 6 illustrates one explanatory method of coupling a flexible strip to an elongated body in accordance with one or more embodiments of the disclosure.

FIG. 7 illustrates another explanatory method of coupling a flexible strip to an elongated body in accordance with one or more embodiments of the disclosure.

FIG. 8 illustrates yet another explanatory method of coupling a flexible strip to an elongated body in accordance with one or more embodiments of the disclosure.

FIG. 9 illustrates another explanatory surgical instrument organizer configured in accordance with one or more embodiments of the disclosure.

FIG. 10 illustrates yet another explanatory surgical instrument organizer configured in accordance with one or more embodiments of the disclosure.

FIG. 11 illustrates yet another explanatory surgical instrument organizer configured in accordance with one or more embodiments of the disclosure.

FIG. 12 illustrates yet another explanatory surgical instrument organizer configured in accordance with one or more embodiments of the disclosure.

FIG. 13 illustrates still another explanatory surgical instrument organizer configured in accordance with one or more embodiments of the disclosure.

FIG. 14 illustrates still another explanatory surgical instrument organizer configured in accordance with one or more embodiments of the disclosure.

FIG. 15 illustrates one step of a method of using one explanatory surgical instrument organizer in accordance with one or more embodiments of the disclosure.

FIG. 16 illustrates one explanatory surgical instrument organizer configured in accordance with one or more embodiments of the disclosure.

FIG. 17 illustrates an explanatory method of using a surgical instrument organizer in accordance with on or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the disclosure provide an elongated body having a flexible strip coupled thereto. In one embodiment, the elongated body is a towel. The flexible strip can be adhesively attached to the towel, stitched to the towel, or disposed within a tubular passage defined within the towel. Other coupling techniques will be obvious to those of ordinary skill in the art having the benefit of this disclosure.

In one embodiment, the flexible strip is bendable to retain the elongated body in a contoured configuration defined by one or more bends of the flexible strip. For example, a health care services provider can apply one or more accordion folds or other folds to the flexible strip to cause the towel to retain those folds.

In one embodiment, the flexible strip is offset from a center of the towel and closer to a first side of the towel than a second, distal side of the towel. Accordingly, a health care services provider can roll the towel into a jellyroll starting with the first end. When the jellyroll is formed, in one embodiment the flexible strip is axially aligned with a center of the jellyroll. Said differently, in one embodiment when the jellyroll is formed the flexible strip is concentrically disposed within a jellyroll of the elongated body. After this rolling step, the health care services provider can bend the flexible strip to apply one or more folds to the flexible strip that are, in turn, retained by the jellyroll. The health care services provider can then place one or more surgical instruments in the one or more folds to reliably and safely hold those surgical instruments in place for use.

Embodiments of the disclosure offer numerous advantages over prior art instrument organizers. A first advantage is that embodiments of the disclosure can easily be formed to different lengths. Embodiments of the disclosure contemplate that each surgical procedure is different and requires different types and numbers of instruments. Prior art towel rolls cannot be configured in different lengths due to the floppiness of the towel. By contrast, the inclusion of the flexible strip in embodiments of the present disclosure allow a resulting jellyroll to be quickly and easily formed to any number of different lengths with different numbers of instrument receiving slots.

Second, the actual rolling process can be simplified. In one or more embodiments, ends of the flexible strip extend beyond edges of the elongated body. Accordingly, when forming a jellyroll, a health care services provider can grasp the ends of the flexible strip to make tighter, quicker, more efficient, and more effective jellyrolls.

Third, embodiments of the disclosure allow health care services providers to use existing skills. Since many health care service providers are experienced in rolling towels, embodiments of the disclosure allow such health care services providers to leverage that skill when using the novel and non-obvious embodiments of the present disclosure.

Fourth, embodiments of the disclosure can be manufactured at very low costs. While some prior art surgical instrument organizers require costly foam, metal, or plastic assemblies, embodiments of the disclosure can require as few as three elements: an elongated body, a flexible strip, and a coupling agent to connect the flexible strip to the elongated body. These components are far less expensive than are other foam or plastic instrument organizers.

Fifth, health care services providers can be assured that each surgical instrument organizer is sterile. While plastic and foam surgical instrument organizers may have to be sanitized between uses, in one embodiment surgical instrument organizers configured in accordance with embodiments of the disclosure are configured to be disposable. Accordingly, the health care services provider can obtain a sterile surgical instrument organizer, use the same, and then simply discard.

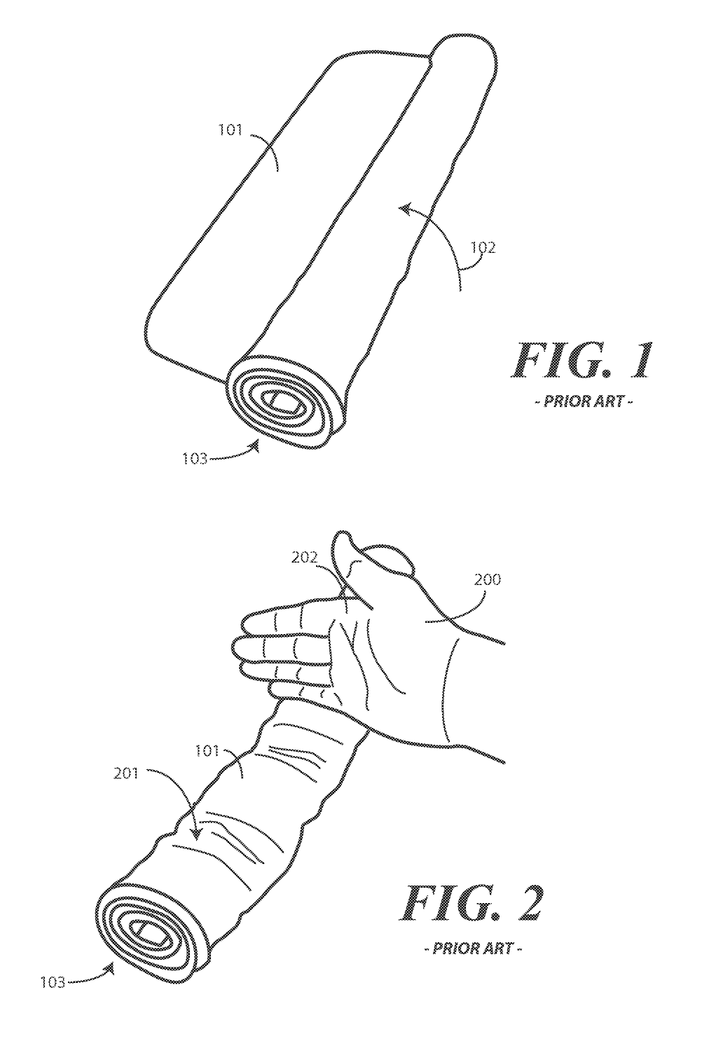

FIGS. 1-3 illustrate a prior art attempt at constructing a surgical instrument holder. Beginning with FIG. 1, a health care services provider first obtains a towel 101. They then roll 102 the towel into a roll that resembles a jellyroll 103. As shown in FIG. 2, the health care services provider 200 then attempts to crimp the jellyroll 103 by chopping it with the hand 202 in a fruitless effort to create some semblance of grooves 201 in the jellyroll 103. This attempt is generally in vain because the towel 101 is generally a cotton towel with no mechanical structure. Thus, not only does the health care services provider 200 run the risk of hurting their hand 202, but they never succeed in effectively shaping the jellyroll 103.

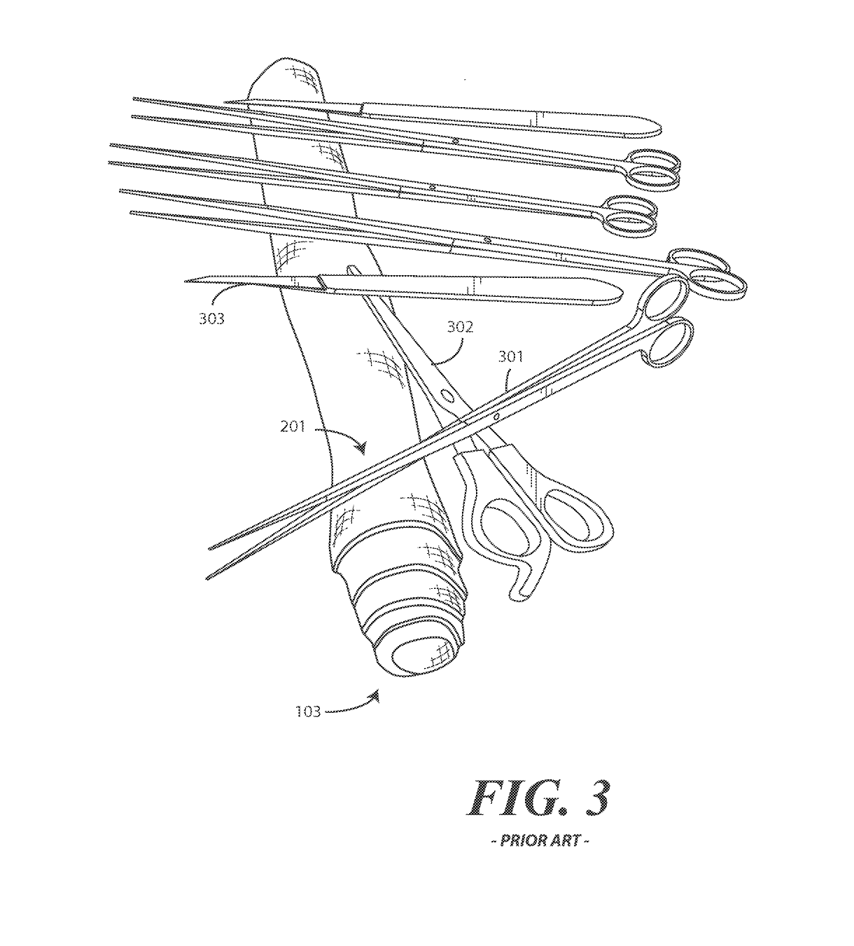

Turning now to FIG. 3, when the process of FIG. 2 is complete, surgical instruments 301 can be placed within the semblance of grooves 201 formed within the jellyroll 103. As shown, the surgical instruments 301 do not have sufficient structure in which to sit and therefore flop over, bump into other instruments 302,303 or slide off the jellyroll 103. This makes locating and grasping the various instruments very difficult. Since the semblance of grooves 201 is just that, a semblance, it is easy to knock the surgical instruments 301 around on the jellyroll 103. As noted above, this can cause them to bump into each other, which makes both identification and selection of the surgical instruments difficult. Where the jellyroll 103 is placed on a Mayo stand, this bumping of the surgical instruments 301 may knock those surgical instruments 301 off the Mayo stand, thereby rendering them non-sterile.

Embodiments of the present disclosure provide a solution to these problems by providing a new and improved surgical instrument organizer. Surgical instrument organizers in accordance with embodiments of the present disclosure have are bendable and retain their shape such that a health care services provider can form the surgical instrument organizer in any shape or length desired, with those folds or bends being reliably retained by the surgical instrument organizer.

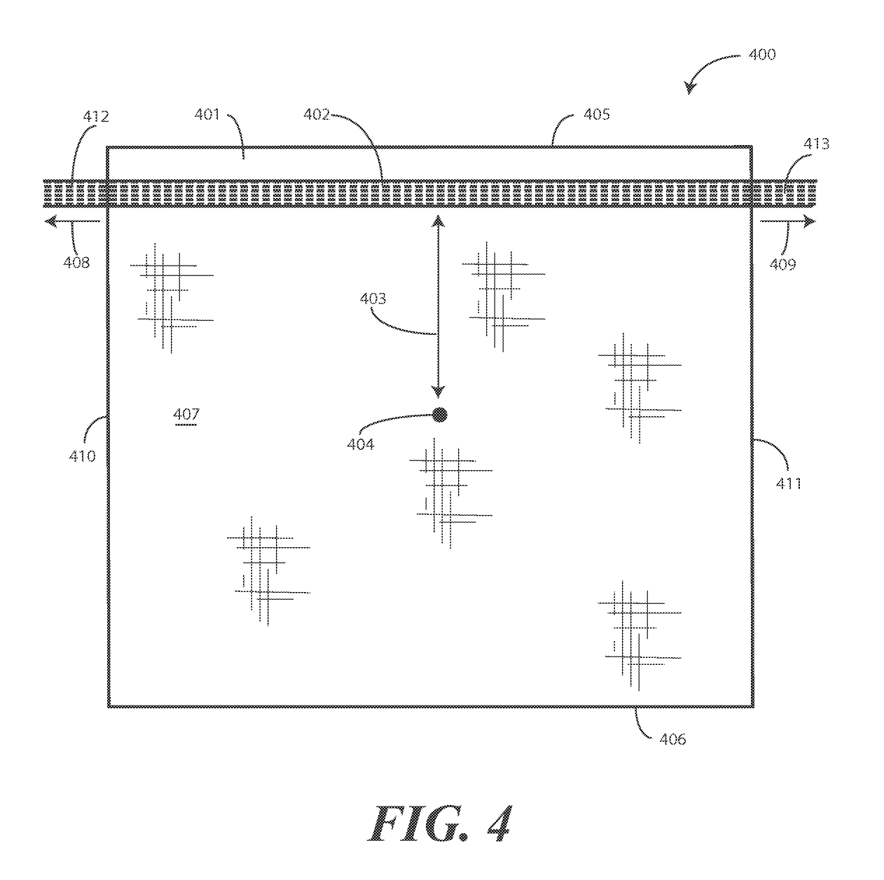

Turning now to FIG. 4, illustrated therein is one explanatory surgical instrument organizer 400 configured in accordance with one or more embodiments of the disclosure. In one embodiment, the surgical instrument organizer 400 comprises an elongated body 401 and a flexible strip 402 coupled to the elongated body. As will be shown and described in more detail below, in one or more embodiments the flexible strip 402 is bendable to retain the elongated body 401—either in a rolled configuration or a flat configuration—in a contoured configuration defined by one or more bends of the flexible strip 402.

In one embodiment, the elongated body 401 is manufactured from a flexible material that allows the elongated body 401 to be folded or rolled. For example, in one embodiment, the elongated body 401 comprises a towel. In one embodiment, the towel is a lint free towel. The use of lint free towels as the elongated body is advantageous in one or more embodiments because, when the elongated body 401 is configured as a surgical instrument organizer, lint will not be transferred from the elongated body 401 to the surgical instruments disposed within the contoured configuration defined by the one or more bends of the flexible strip 402. This is advantageous because it prevents the introduction of fibers from the elongated body 401 into a surgical site on a patient when the surgical instruments are used. This advantageously helps to prevent infection and promotes surgical site healing.

In one or more embodiments, both the elongated body 401 and the flexible strip 402 are sterilized. Accordingly, when a health care services provider needs to use the surgical instrument organizer 400, no sanitization or sterilization is required. In one embodiment, the elongated body 401 and flexible strip 402 are packaged in a sterile packaging so that they may be opened in a sterile condition.

In one embodiment, the elongated body 401 comprises a lint free cotton towel. For example, in one embodiment the elongated body 401 is manufactured from a non-woven cotton fabric. For example, the elongated body 401 may be manufactured form non-woven layers of laminated cotton material. In other embodiments, the elongated body may be manufactured from synthetic materials. For instance, in another embodiment the elongated body 401 can be manufactured from a flexible thermoplastic material. In still other embodiments, the elongated body 401 can be manufactured from a material that is combustible to facilitate disposal via incineration to avoid cross contamination of bacteria or pathogens. Further, in one or more embodiments additives can be included in the material of the elongated body 401, such as odor-controlling additives, anti-microbial additives, and so forth.

In still other embodiments, the elongated body 401 may be a multi-ply assembly. For example, the elongated body 401 may comprise a pile stack with some piles manufactured from first materials, while other piles are manufactured from other materials. Examples of such materials include tissue, thermoplastic non-woven webs, and so forth. Still other materials suitable for use as the elongated body 401 will be obvious to those of ordinary skill in the art having the benefit of this disclosure.

In one embodiment, the elongated body 401 is color coded to indicate that it is to be used as a surgical instrument organizer. For example, in one embodiment the elongated body 401 is colored white so as to indicate it is to be used as a surgical instrument organizer. The color could be used to indicate other characteristics as well, including the fact that the flexible strip 402 included, or that the elongated body 401 is sterile and suitable for use in an operating room. In other embodiments, the elongated body 401 may have no color at all.

In the illustrative embodiment of FIG. 1, the elongated body 401 is rectangular in shape when viewed in the plan view shown in the figure. However, it should be noted that the elongated body 401 can take other shapes as well. For example, the elongated body 401 can be round in plan view, ovular in plan view, hexagonal in plan view, or take freeform shapes in plan view. Still other shapes will be obvious to those of ordinary skill in the art having the benefit of this disclosure. The elongated body 401 can be made in any of a number of sizes and shapes. In one embodiment, the elongated body 401 has a length of about sixteen inches and a width of about seventeen inches. The term “about” is used to indicate a dimension inclusive of manufacturing tolerances. For example, if the manufacturing tolerances were plus or minus one-half inch, “about seventeen inches” would include a range of 16.5 inches to 17.5 inches, and so forth.

The elongated body 401 defines a first major face 407 and a second major face (not shown in FIG. 1) disposed opposite the first major face 407. In this illustrative embodiment, the flexible strip 402 is disposed along the first major face 407 of the elongated body 401.

In the illustrative embodiment of FIG. 1, the flexible strip 402 is offset 403 from a center 404 of the elongated body 401 so as to be closer to a first side 405 than to a second, distal side 406 of the elongated body 401. The second, distal side 406 is “distal” from the first side 405 because the center 404 of the elongated body 401 is disposed between the first side 405 and the second, distal side 406.

In this illustrative embodiment, the flexible strip 402 extends 408,409 beyond at least one edge of the elongated body 401. The flexible strip 402 of this embodiment extends 408,409 beyond two edges of the major face 407 of the elongated body 401. Specifically, it extends beyond both a first edge 410 and a second edge 411 of the elongated body 401 in this embodiment.

In one embodiment, the flexible strip 402 of this embodiment extends 408,409 approximately one inch beyond the first edge 410 and the second edge 411 of the elongated body 401. Advantageously, when the elongated body is rolled into a jellyroll, a first end 412 and a second end 413 of the flexible strip 402 can be grasped to make the rolling process easier. Additionally, once the jellyroll is formed, the first end 412 and the second end 413 can be grasped to make bending or applying folds to the flexible strip 402 easier.

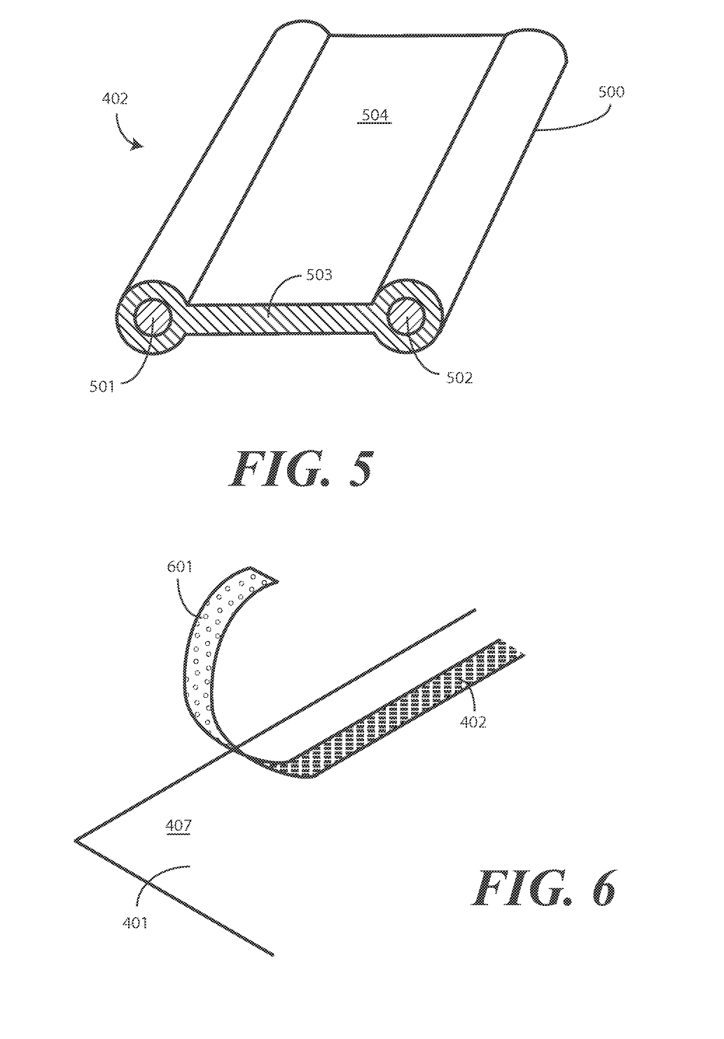

The flexible strip 402 can take a variety of forms. In one or more embodiments, the flexible strip 402 includes one or more wires or other bendable pieces of metal so as to be easily deformable to a desired shape. In one embodiment, the flexible strip 402 comprises a two-wire strip. While a two-wire strip will be used as an explanatory flexible strip 402 for illustrative purposes, it should be noted that the flexible strip 402 could include one wire, three wires, or more wires. Turning to FIG. 5, illustrated therein is one example of such a two-wire strip 500.

As shown in FIG. 5, in one embodiment the flexible strip 402 is a two-wire strip 500 comprising two strands 501,502 of flexible wire disposed within a thermoplastic binder 503. In one embodiment, the flexible wire is a bendy wire manufactured from a malleable metal such as soft-annealed iron having a twenty-six or greater gauge measurement. Such wires are less than ten thousandths of an inch in diameter. Thicker wires can be used to stiffen the flexible strip 402. Moreover, in other embodiments the flexible strip 402 can be a single-wire strip, three-wire strip, four-wire strip, and so forth. In other embodiments, such as where the surgical instrument organizer (400) will be subjected to radiation to kill microorganisms, bacteria, or pathogens, non-metallic malleable materials can be substituted for the wires described above. Still other configurations will be obvious to those of ordinary skill in the art having the benefit of this disclosure.

The thermoplastic binder 503 can be made from a variety of materials. In one or more embodiments, the thermoplastic binder 503 is manufactured from a flexible thermoplastic. Examples of flexible thermoplastics include polyvinyl materials, polystyrene, nylon, cellulose esters, and so forth. Polyethylene can also be used for the thermoplastic binder 503. In one or more embodiments the material selected for use as the thermoplastic binder 503 is one that securely bonds to the strands 501,502.

In one or more embodiments, the center 504 of the two-wire strip 500 can be textured.

For example, it can include one or more bumps, protrusions, slits, or other surface features that assist a user in grasping the two-wire strip 500 during a bending operation. Further, the thermoplastic binder 503 can be color-coded to indicate, for example, that the elongated body (401) is specifically designed as a surgical instrument organizer (400).

In one or more embodiments, the two-wire strip 500 is manufactured via an extrusion process. In one explanatory extrusion process, the strands 501,502 are passed through an extruder that applies the thermoplastic binder 503 about the strands 501,502.

The flexible strip 402 can be attached to the elongated body (401) in a variety of ways.

FIGS. 6-8 illustrate three examples of ways that the flexible strip 402 can be coupled to the elongated body (401). Still others will be obvious to those of ordinary skill in the art having the benefit of this disclosure.

Beginning with FIG. 6, in this embodiment the flexible strip 402 is adhesively coupled to the elongated body 401. For example, a layer 601 of adhesive can be applied to a first side of the flexible strip 402. Optionally, a releasable binder can be attached to the layer 601 of adhesive to prevent the adhesive from sticking to anything until desired. Where the releasable binder is included, it can be removed. The exposed layer 601 of adhesive can then be pressed against the major face 407 of the elongated body 401 to couple the flexible strip 402 to the elongated body 401.

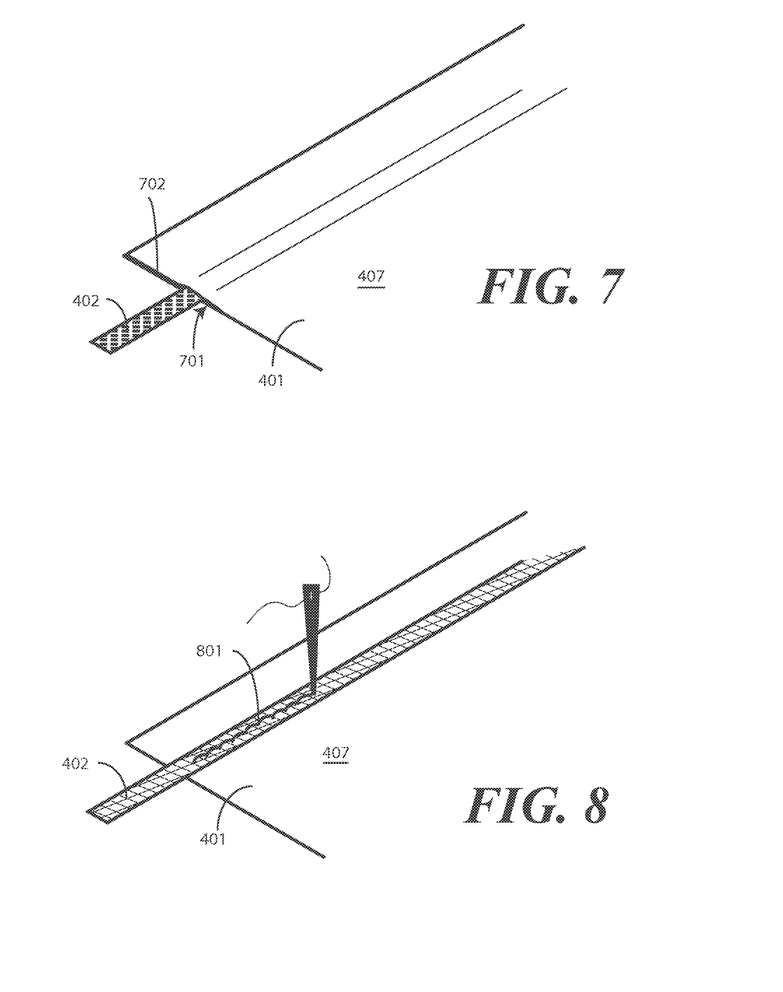

Turning now to FIG. 7, in this embodiment the elongated body 401 defines a tubular passage 701. The flexible strip 402 is then disposed within the tubular passage 701. For example, a layer 702 of the elongated body 401 may fold back over the major face 407 and be coupled thereto to define the tubular passage 701. Alternatively, a second layer may be attached to the major face 407 to define the tubular passage 701. Where the elongated body 401 is a multi-ply structure, the tubular passage 701 may be defined between various piles or layers. Still other methods for creating the tubular passage 701 will be obvious to those of ordinary skill in the art having the benefit of this disclosure. Note that the flexible strip 402 may optionally be adhesively—or by other means—retained within the tubular passage 701.

Turning to FIG. 8, in this embodiment the flexible strip 402 is coupled to the major face 407 of the elongated body 401 by stitching 801. For example, in one embodiment the binder of the flexible strip 402 can be fabric or another material that can be stitched to the elongated body 401. As noted above, the methods shown in FIGS. 6-8 are meant as illustrative examples. Others will be obvious to those of ordinary skill in the art having the benefit of this disclosure.

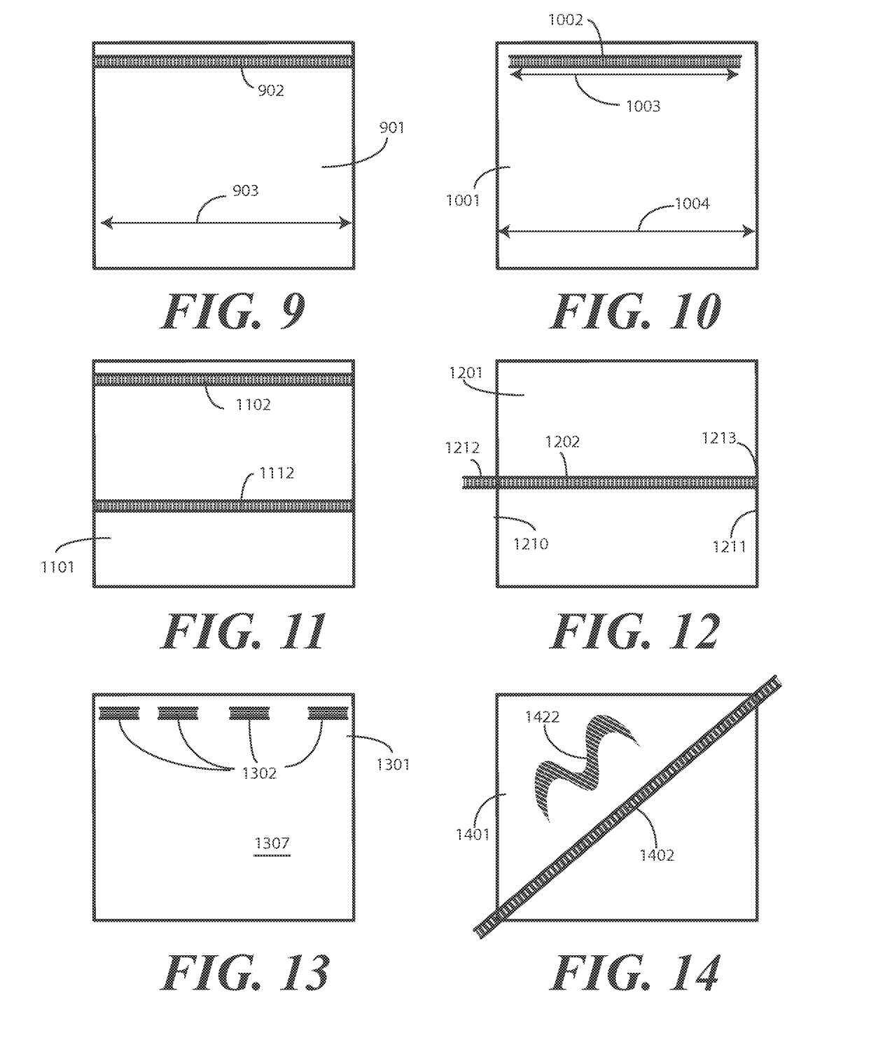

Turning now to FIGS. 9-14, illustrated therein are examples of variations of surgical instrument organizers included within embodiments of the disclosure. Beginning with FIG. 9, in this embodiment the flexible strip 902 has a width 903 that is equivalent to that of the elongated body 901. Recall from above that in the embodiment of FIG. 4, the flexible strip (402) was wider than the elongated body (401). By contrast, in FIG. 9, the flexible strip 902 and elongated body 901 have a common width 903.

In FIG. 10, the flexible strip 1002 has a width 1003 that is shorter than the width 1004 of the elongated body 1001. Advantageously, this configuration offers the benefit of fully concealing the flexible strip 1002 in the interior of the jellyroll when the elongated body 1001 is rolled.

In FIG. 11, two flexible strips 1102,1122 are coupled to the elongated body 1101. While only one flexible strip (402) was coupled to the elongated body (401) of FIG. 4, embodiments of the disclosure are not so limited. Two flexible strips 1102,1122 can be coupled to the elongated body 1101, as shown in FIG. 11. Alternatively, three, four, or more flexible strips can be used as well. The addition of more than one flexible strip advantageously allows the stiffness of the mechanical support offered by the flexible strips 1102,1122 to be adjusted to a desired level.

In FIG. 12, the flexible strip 1202 is centrally disposed along the elongated body 1201.

Additionally, the flexible strip 1202 of FIG. 12 includes only one end 1212 that extends beyond an edge 1210 of the elongated body 1201. The other end 1213 of this embodiment is co-extensive with the other edge 1211 of the elongated body 1201. In other embodiments, the other end 1213 of the flexible strip 1202 can terminate short of the other end 1213 of the elongated body 1201.

In FIG. 13, the flexible strip 1302 comprises a plurality of flexible strip segments. In this embodiment, each segment is separated from other segments along a major face 1307 of the elongated body 1301.

In FIG. 14, two embodiments are shown. First, a flexible strip 1402 is disposed diagonally across the elongated body 1401. Another flexible strip 1422 is non-linear in shape. It should be noted that any of the embodiments of FIG. 4 or FIGS. 9-14 can be used in combination where desired. Still other embodiments will be obvious to those of ordinary skill in the art having the benefit of this disclosure.

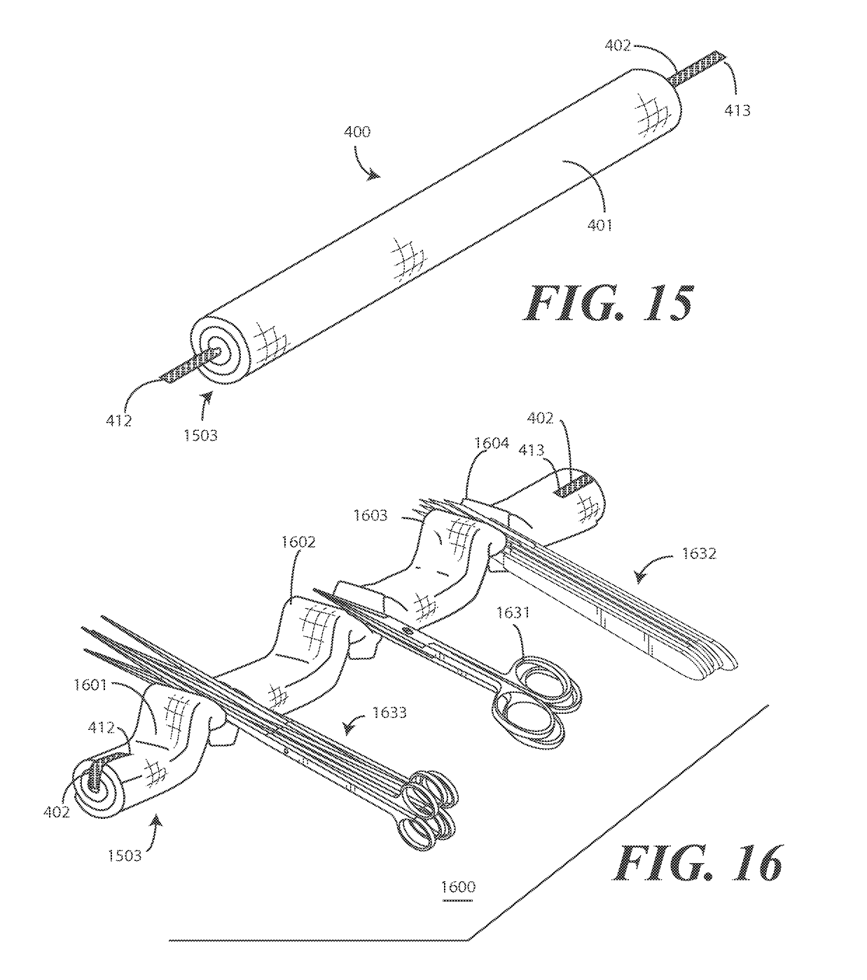

Turning now to FIGS. 15-16, illustrated therein are steps of a method of using a surgical instrument organizer configured in accordance with one or more embodiments of the disclosure. Beginning with FIG. 15, a surgical instrument organizer 400 has been obtained. In this embodiment, the surgical instrument organizer 400 comprises an elongated body 401 and a flexible strip 402 coupled to the elongated body 401. In this embodiment, the flexible strip 402 is bendable to retain the elongated body 401 in a contoured configuration. As shown in FIG. 15, the elongated body 401 has been rolled about the flexible strip 402 to form a jellyroll 1503. In one embodiment, this rolling step comprises grasping the ends 412-413 of the flexible strip 402. In this embodiment, the flexible strip 402 is concentrically disposed within the roll of the elongated body 401.

Turning to FIG. 16, one or more folds 1601,1602,1603,1604 have been applied to the flexible strip 402 by bending the flexible strip 402. Additionally, the ends 412,413 of the bendable strip have advantageously been folded back over the ends of the jellyroll 1503 to keep it from unraveling. In one embodiment, the flexible strip 402 is malleable so as to be adjustable into different shapes. Accordingly, a user can bend the flexible strip 402 such that it takes any of a variety of shapes, including semi-circular, angular, elliptical, or free form shapes. In one embodiment, these folds 1601,1602,1603,1604 comprise an accordion fold. For example, as shown in FIG. 16, in one embodiment the elongated body 401 comprises a first accordion fold 1601 and a second accordion fold 1602. At least one surgical instrument 1631 can be placed within these accordion folds. Other fold configurations will be obvious to those of ordinary skill in the art having the benefit of this disclosure.

Accordingly, the surgical instrument organizer 400 has been formed into a desired shape for a particular surgical procedure so as to accommodate only those surgical instruments required for the particular surgical procedure. The flexible strip 402 ensures that the surgical instrument organizer 400 reliably remains in this contoured configuration defined by one or more bends of the flexible strip 402.

In the embodiment of FIG. 16, the surgical instrument organizer 400 has been placed on a horizontal surface 1600, such as a Mayo cart. As shown, an end of the surgical instrument 1631 has been placed into the one or more folds 1601,1602,1603,1604. The surgical instrument 1631 remains securely held within the folds and easily graspable by a surgeon or other health care services provider. Additionally, other surgical instruments 1632,1633 can be arranged neatly in groups. These instruments will not topple, slide off the jellyroll 1503, or bump into other instruments.

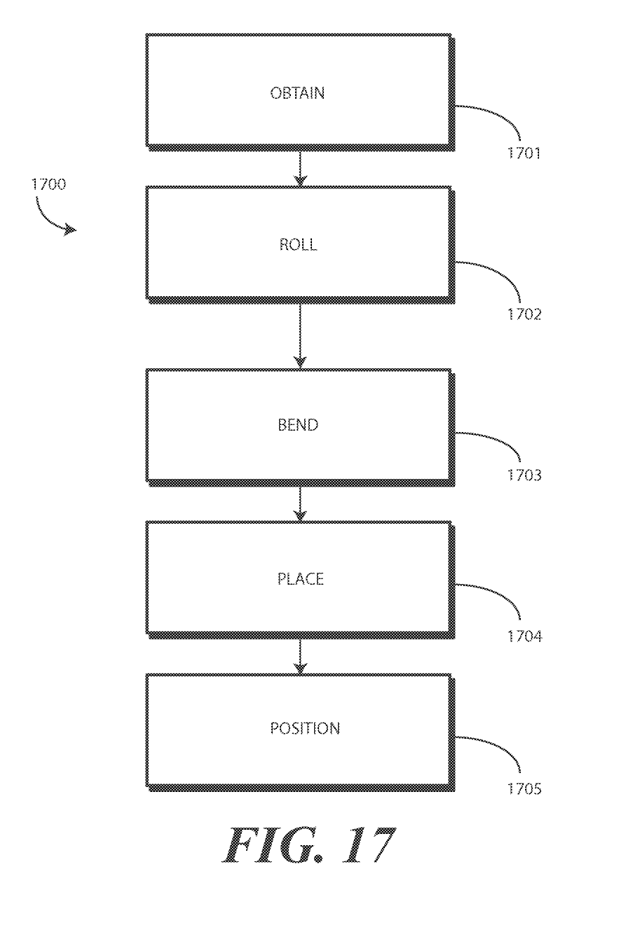

A summary of this method is shown in FIG. 17. Turning now to FIG. 17, illustrated therein is a flow chart of a method 1700 of organizing surgical instruments. At step 1701, the method 1700 includes obtaining a surgical instrument organizer. In one embodiment, the surgical instrument comprises an elongated body and a flexible strip coupled to the elongated body. In one embodiment, the elongated body is a towel. The flexible strip is bendable to retain the elongated body in a contoured configuration.

At step 1702, the method 1700 includes rolling the elongated body about the flexible strip. In one embodiment, this step 1702 includes grasping ends of the flexible strip extending beyond edges of the elongated body. In one embodiment, this step 1702 result sin the flexible strip being concentrically disposed within a roll of the elongated body.

At step 1703, the method 1700 includes bending the flexible strip to apply one or more folds to the flexible strip. At step 1704, the method 1700 optionally includes placing the surgical instrument organizer on a horizontal surface after the bending. At step 1705, the method includes positioning a surgical instrument in the one or more folds.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A surgical instrument organizer, comprising:
an elongated body; and
a flexible strip coupled to the elongated body;
the flexible strip bendable to retain the elongated body in a contoured configuration defined by one or more bends of the flexible strip.

2. The surgical instrument organizer of claim 1, wherein the elongated body comprises a towel.

3. The surgical instrument organizer of claim 2, the one or more bends comprising one or more accordion folds.

4. The surgical instrument organizer of claim 3, further comprising at least one surgical instrument positioned within the one or more accordion folds.

5. The surgical instrument organizer of claim 2, the flexible strip offset from a center of the towel and closer to a first side of the towel than a second, distal side of the towel.

6. The surgical instrument organizer of claim 5, the flexible strip disposed along a major face of the towel.

7. The surgical instrument organizer of claim 6, the flexible strip extending beyond at least one edge of the major face of the towel.

8. The surgical instrument organizer of claim 7, the flexible strip extending beyond two edges of the major face of the towel.

9. The surgical instrument organizer of claim 8, the flexible strip comprising a two-wire flexible strip.

10. The surgical instrument organizer of claim 8, the flexible strip adhesively coupled to the major face of the towel.

11. The surgical instrument organizer of claim 8, the flexible strip coupled to the major face of the towel by stitching.

12. The surgical instrument organizer of claim 2, the flexible strip comprising a plurality of flexible strip segments, each segment separated from other segments along a major face of the towel.

13. The surgical instrument organizer of claim 1, the elongated body defining a tubular passage, the flexible strip disposed within the tubular passage.

14. The surgical instrument organizer of claim 1, the flexible strip disposed diagonally along a major face of the elongated body.

15. A method of organizing surgical instruments, the method comprising:
obtaining a surgical instrument organizer comprising an elongated body and a flexible strip coupled to the elongated body, the flexible strip bendable to retain the elongated body in a contoured configuration;
rolling the elongated body about the flexible strip; and
bending the flexible strip to apply one or more folds to the flexible strip.

16. The method of claim 15, further comprising placing the surgical instrument organizer on a horizontal surface after the bending.

17. The method of claim 16, further comprising positioning a surgical instrument in the one or more folds.

18. The method of claim 15, the rolling resulting in the flexible strip being concentrically disposed within a roll of the elongated body.

19. The method of claim 15, wherein the elongated body comprises a towel.

20. The method of claim 19, wherein the rolling comprises grasping ends of the flexible strip extending beyond edges of the towel.
